(19) United States
(12) Patent Application Publication
(10) Pub. No.: US 2005/0245840 A1
(43) Pub. Date: Nov. 3, 2005

(54) Implantable urinary tract monitor

(75) Inventors: Mark A. Christopherson, Shoreview, MN (US); Warren L. Starkebaum, Plymouth, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/833,776

(22) Filed: Apr. 28, 2004

(51) Int. Cl. A61B 5/00; A61K 31/54

(52) U.S. Cl. 600/561; 600/365; 128/903; 600/309

(57) ABSTRACT

An implantable urinary tract monitor is configured for indwelling urodynamic testing, indwelling urinalysis, or both. A urinary tract monitor in accordance with the invention incorporates a fixation structure to selectively position the monitor at a tissue site within the bladder or urethra. In this manner, the monitor is implanted within the patient and, if desired, can accompany the patient throughout a routine of normal daily activities.

### FIELD OF THE INVENTION

The invention relates to medical sensors and, more particularly, sensors for sensing physiological conditions within a urinary tract.

### BACKGROUND

One form of urinary tract analysis is urodynamic testing. Many people suffer from involuntary urine leakage, i.e., urinary incontinence. Others may suffer from blocked or restricted urine flow. Other urinary disorders include frequent urination, sudden urges to urinate, problems starting a urine stream, painful urination, problems emptying the bladder completely, and recurrent urinary tract infections. A physician uses a urodynamic test to study how a patient stores and releases urine.

Different muscles, nerves, organs and conduits within the urinary tract cooperate to collect, store and release urine. A variety of disorders may compromise the urinary tract performance and contribute to incontinence or restricted flow. Many of the disorders may be associated with aging, injury or illness. For example, benign prostate hyperplasia (BPH) may create an occlusion of the male urethra due to prostate enlargement, and cause blocked or restricted urine flow. On the other hand, aging can often result in weakened sphincter muscles, which cause incontinence, or weakened bladder muscles, which prevent complete emptying.

A urodynamic test reveals how well the bladder and sphincter muscles perform, and may help identify the causes of various urinary tract disorders. Urodynamic testing can take the form of simple observation or precise measurement using monitors that sense physiological conditions such as urine pressure, flow, velocity, volume, and the like. Some monitors sense the occurrence and force of bladder contractions to identify abnormal bladder function. Other monitors may determine a volume of urine remaining in the bladder following urination. Hence, urodynamic testing may focus on the ability of the bladder to empty steadily and completely.

Another form of urinary tract analysis is urinalysis. Urinalysis typically involves diagnostic analysis of a urine sample, e.g., by chemical, physical or microscopic examination of a urine specimen. Urinalysis may be used to identify diseases or disorders of the kidneys or urinary tract, monitor diabetic patients, detect drug abuse, or test for pregnancy. In some cases, urinalysis may be used to detect odor, color, or chemical content of a urine sample. As examples, urinalysis may focus on urine acidity or the presence of sugar, proteins, blood, keytones, bilirubin or other substances such as bacteria, yeast cells or parasites in the urine.

Urodynamic testing ordinarily requires catheterization of the patient in order to place a monitor within the bladder or urethra. For this reason, urodynamic testing typically takes place within a clinical setting. In some cases, the presence of a catheter can disrupt the normal physiological function of the urinary tract. Although ambulatory catheterization is possible, it can be uncomfortable and may obtain measurements that are not be representative of normal physiological function. In addition, the urinary catheter can be uncomfortable for the patient.

Urinalysis ordinarily requires bladder catheterization or capture of a voided urine sample. In each case, the urine sample represents the state of the patient's urine at a particular time. Often, urine samples must be collected and stored until a clinical laboratory can analyze the samples. Accordingly, the urinalysis results may be delayed. Also, to reevaluate the patient's urine, catheterization must be maintained or repeated, or samples must be repeatedly collected over time.

Various urodynamic testing systems are described in U.S. Pat. No. 4,873,990 to Holmes et al., U.S. Pat. No. 5,331,548 to Rollema et al., U.S. Pat. No. 6,454,720 to Clerc et al. Siwapomsathain et al. describes a bladder monitor with wireless telemetry in Siwapomsathain et al., “A Telemetry and Sensor Platform for Ambulatory Urodynamics,” Department of Electrical and Computer Engineering, University of Wisconsin—Madison. J. Coosemans et al. describes an implantable bladder pressure monitor with wireless telemetry in “Datalogger for Bladder Pressure Monitoring With Wireless Power and Data Transmission,” Katholieke Universiteit Leuven, Department ESAT-MICAS, Belgium, Belgian Day on Biomedical Engineering, 2003. U.S. Pat. No. 5,704,353 to Kalb et al. describes a urinary diagnostic catheter for analyzing various chemical and physical properties of urine in the bladder.

Table 1 below lists documents that disclose various techniques for urodynamic testing and urinalysis.

TABLE 1

| Patent Number | Inventors/Author | Title |
|---|---|---|
| 4,873,990 | Holmes et al. | Circumferential Pressure Probe |
| 5,331,548 | Rollema et al. | Method and system for |
| | | on-line measurement, |
| | | storage, retrieval and |
| | | analysis of urodynamical |
| | | data |
| 6,454,720 | Clerc et al. | System for measuring |
| | | physical parameters |
| | | with a medical probe |
| 5,704,383 | Kalb et al. | Urinary diagnostic catheter |
| Not applicable | J. Coosemans et al. | Datalogger for Bladder |
| | | Pressure Monitoring |
| | | With Wireless Power |
| | | and Data Transmission |
| Not Applicable | Siwapomsathain et | A Telemetry and Sensor Platform |
| | al. | for Ambulatory Urodynamics |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY OF THE INVENTION

In general, the invention is directed to an implantable urinary tract monitor. The monitor is configured for indwelling urodynamic testing, indwelling urinalysis, or both. A urinary tract monitor in accordance with the invention incorporates a fixation structure to selectively position the monitor at a tissue site within the bladder or urethra. In this manner, the monitor is implanted within the patient and, if desired, can accompany the patient throughout a routine of normal daily activities.

Various embodiments of the present invention provide solutions to one or more problems existing in the prior art with respect to prior techniques for urodynamic testing or urinalysis. These problems include the need for persistent catheterization to perform urodynamic testing, or catheterization to obtain a urine sample, or the capture of a voided urine sample. Additional problems relate to the need for repeated catheterization or repeated capture of samples for further urodynamic testing or urinalysis. As further problems, existing techniques for urodynamic testing or urinalysis may cause patient discomfort, and alter the physiological function of the patient's urinary tract. In addition, urodynamic testing and especially urinalysis may suffer from delays between catheterization or sample-taking and generation of results. In addition, other problems relate to the inability to track urodynamic conditions or perform urinalysis continuously or over an extended period of time, especially as the patient goes about his or her daily routine. Instead, existing techniques are often restricted to hospital stays, clinical visits, or individual samples, and therefore produce results for limited sets of data points.

Various embodiments of the present invention are capable of solving at least some of the foregoing problems. When embodied in an implantable urinary tract monitor, for example, the invention includes a variety of features that facilitate urodynamic testing or urinalysis with an implantable monitor. The monitor is configured as an indwelling device, which may be positioned within the bladder or urethra for an extended period of time, on either a temporary or chronic basis. In this manner, the monitor can sense urodynamic parameters or urine characteristics on a continuous basis. The monitor may be placed with a catheter, cystoscope, or the like, and does not require persistent catheterization. Also, the monitor may provide built-in processing or cooperate with an external receiver with processing capabilities to reduce delays between analysis and generation of results. The implanted monitor may accompany a patient throughout a routine of daily activities, if desired, to track urodynamic conditions or perform urinalysis continuously or over an extended period of time.

Various embodiments of the invention may possess one or more features to solve the aforementioned problems in the existing art. In some embodiments, a urinary tract monitor for placement within the bladder or urethra includes a sensor, a telemetry unit, a power source, a device housing, and a fixation mechanism. The device housing is sized for introduction into the urethra. The fixation mechanism positions the device housing within the bladder or urethra. The fixation mechanism may take a variety of forms, including a pin or shaft that penetrates or pinches tissue within the bladder or urethra, or a stent-like frame that is expandable to engage the walls of the urethra and thereby hold the monitor in place. Other possible fixation mechanisms include suction devices, magnetic devices, or helical screw-like mechanisms.

An external receiver may be provided to obtain urodynamic or urinalysis information from the implanted monitor by wireless telemetry. In some embodiments, the monitor or the receiver may generate a control signal based on the urodynamic or urinalysis information to activate an advisory, or activate or adjust a therapy applied to the patient. For example, the advisory may indicate a need for intake of a pharmaceutical or other substance by the patient to moderate the level of a parameter detected by urodynamic testing or urinalysis. As another example, the control signal may be applied to adjust an electrical stimulation signal to control incontinence or adjust a dosage of a medicament delivered by an external or implanted pump.

In comparison to known techniques for urodynamic testing or urinalysis, various embodiments of the invention may provide one or more advantages. For example, an implantable urinary tract monitor permits urodynamic testing or urinalysis to be performed on a substantially continuous basis, if desired, without the need for persistent catheterization in the case of urodynamic testing, or repeated catheterizations or repeated voided urine collection to obtain urine samples in the case of urinalysis. In addition, following initial placement with a catheter, cystoscope, or the like, there is no need for persistent catheterization, eliminating significant discomfort and reducing the impact on normal physiological function of the patient's urinary tract. Also, the monitor may reduce delays between analysis and generation of results, and accompany a patient throughout a routine of daily activities.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating an implantable urinary tract monitor shown in conjunction with the bladder and urethra of a patient.

FIG. 2 is a functional block diagram illustrating a urinary tract monitor.

FIG. 3 is a functional block diagram illustrating an external receiver for communication with the urinary tract monitor of FIG. 2.

FIG. 4 is a functional block diagram illustrating a network for communication of information obtained by urinal tract monitors.

FIG. 5 is a cross-sectional side view of a urinary tract monitor attached to a tissue site within the bladder or urethra.

FIG. 6 is a schematic diagram illustrating deployment of the monitor of FIG. 4 within a patient's urinary tract with an endoscopic delivery device.

FIG. 7 a schematic diagram illustrating further deployment of the monitor of FIG. 4 within a patient's urinary tract with an endoscopic delivery device.

FIG. 8 is a cross-sectional side view of the distal end of the urinary tract monitor and endoscopic delivery device of FIGS. 6 and 7.

FIG. 9 is a side view of a monitor with a fixation structure in the form of an expandable frame.

FIG. 10A is a cross-sectional view of the monitor and expandable frame of FIG. 9 in an unexpanded state within the urethra.

FIG. 10B is a cross-sectional view of the monitor and expandable frame of FIG. 9 in an expanded state within the urethra.

FIG. 11 is another side view of the monitor and expandable frame of FIG. 9 positioned within the urethra.

FIG. 12 is a cross-section view of an alternative monitor mounted to an expandable frame.

FIG. 13 is flow diagram illustrating a method for placement and use of an implantable urinary tract monitor.

FIG. 14 is a functional block diagram illustrating communication of information from an implantable urinary tract monitor to an external receiver to control a therapy device.

FIG. 15 is a flow diagram illustrating communication of information from an implantable urinary tract monitor to an external receiver to control a therapy device.

FIG. 16 is a flow diagram illustrating communication of information from an implantable urinary tract monitor to an external receiver to generate advisories.

FIG. 17 is a conceptual diagram of an external receiver equipped to communicate an advisory with respect to a sensed condition.

FIG. 18 is a conceptual diagram of another external receiver equipped to communicate an advisory with respect to a sensed condition.

### DESCRIPTION OF THE PREFERRED EMBODIMENTS

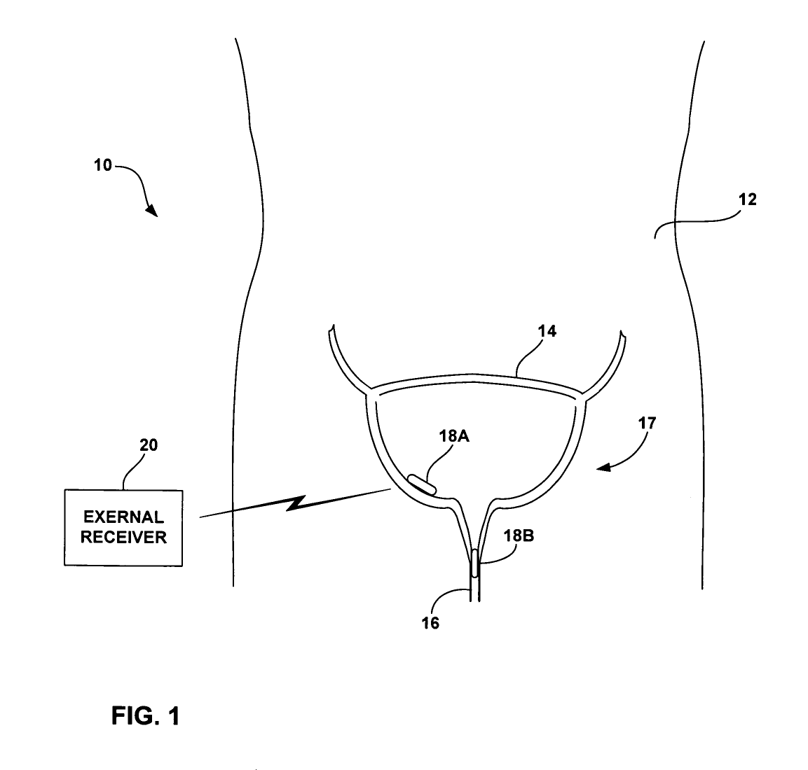

FIG. 1 is a schematic diagram illustrating an implantable urinary tract monitor system 10 shown in conjunction with a patient 12 and, in particular, a patient bladder 14 and urethra 16 forming part of the patient's urinary tract 17. As shown in FIG. 1, system 10 includes an implanted monitor 18A or 18B and an external receiver 20. Monitor 18A is shown at a target location within bladder 14, and monitor 18B is shown at a target location within urethra 16. One or more urinary tract monitors 18A, 18B may be placed within urinary tract 17. However, two monitors 18A, 18B are shown in FIG. 1 primarily to depict different placement positions for a single monitor, rather than the use of multiple monitors, although multiple monitors are possible. Monitors 18A, 18B will be generally referred to herein collectively as monitor 18.

An implantable urinary tract monitor 18 may be configured to sense one or more physiological conditions within urinary tract 17. For example, the physiological conditions may include one or more urodynamic conditions such as urine pressure, urine volume, urine flow, urine pH, temperature, bladder contraction, or urinary sphincter contraction. Hence, in some embodiments, monitor 18 is designed to perform indwelling urodynamic tests without the need for presence of a catheter within the urethra. Alternatively, the physiological conditions may include one or more physical characteristics of urine in urinary tract 17, such as presence of drug residue, sugar, proteins, blood, keytones, bilirubin, bacteria, yeast cells, and parasites in the urine. Also, monitor 18 may be configured to sense levels of the physical characteristics, such as glucose levels. In this case, monitor 18 is designed to perform indwelling urinalysis without the need for a urethral catheter.

Monitor 18 may be placed at a target location within urinary tract 17 by endoscopic delivery, e.g., using a catheter, cystoscope, endoscope, or the like. Monitor 18 may be implanted temporarily or chronically. The target location may be within bladder 14 or within urethra 16. As will be described, monitor 18 may include a fixation structure to securely position the monitor at a target tissue location within urinary tract 17. Upon fixation of monitor 18, the endoscopic delivery device may be withdrawn from urinary tract 17 of patient 12. In this manner, monitor 18 can remain in a desired position for an extended period of time, avoiding the need for recatheterization for additional urodynamic testing, or recatheterization or repeated collection of urine samples for additional urinalysis. In addtion, monitor 18 may accompany patient 12 outside the clinic and throughout a routine of daily activities. This aspect may offer a better physiological representation of urinary tract function than techniques that require a catheter and can only be used for a short observation period.

Monitor 18 is delivered via the urethra, and thereby requires no surgical procedures. Monitor 18 may be placed within bladder 14 or within urethra 16, depending on the desired urodynamic or urinalysis application for which the monitor is configured. In either case, monitor 18 obtains information and transmits the information to an external receiver 20. Alternatively, monitor 18 may include internal memory to store information for recovery after the monitor has been removed from urinary tract 17. In each case, monitor 18 is capable of continuously or periodically performing urodynamic testing or urinalysis over an extended period of time. In addition, in some embodiments, monitor 18 may sense conditions and provide instantaneous feedback via transmission to external receiver 20.

As further shown in FIG. 1, monitor 18 may have a capsule-like device housing sized for endoscopic introduction via urethra 16. For example, the capsule-like device housing of monitor 18 may have a maximum length of less than approximately 15 mm and a maximum width of less than approximately 5 mm, although smaller dimensions may be desirable given the diameter of urethra 16. In some embodiments, the capsule-like device housing may be substantially cylindrical, with a length greater than its diameter and flat or rounded ends, although the invention is not limited to any particular shape. For a cylindrical device housing, monitor 18 may have a maximum height of less than approximately 15 mm and a maximum diameter of less than approximately 5 mm. The device housing may be formed from a variety of biocompatible materials such as stainless steel or titanium. Alternatively, components associated with monitor 18 may be encapsulated in silicone or other biocompatible materials.

The capsule-like device housing of monitor 18 includes a sensor configured to sense particular physiological conditions in support of urodynamic testing, urinalysis, or both. The monitor housing further includes a power source, a telemetry unit, signal processing electronics, and the fixation structure. Again, the fixation structure secures monitor 18 to a target location within bladder 14 or urethra 16. In particular, the fixation structure may perforate a mucosal lining within urinary tract 17 tract, or grip or “pinch” a fold of the mucosal lining. To place monitor 18, a distal end of an endoscopic delivery device is inserted into urethra 16 and guided to a target location within the urinary tract. Upon arrival at the desired location, which may be viewed by external or endoscopic imaging, the fixation structure is activated to secure monitor 18 in place.

Following placement of monitor 18, the endoscopic delivery device is withdrawn from patient 12. Accordingly, there are no catheters, leads or other connections that extend outside of patient 12. On the contrary, monitor 18 may be entirely self-contained, self-powered and integrated within a common, capsule-like housing. In some embodiments, an external source of inductively coupled power may be used to power some features of monitor 18. For example, monitor 18 may include an inductive power interface for transcutaneous inductive power transfer to power higher energy functions such as telemetry. However, monitor 18 typically will include a small battery cell within the capsule-like monitor housing.

The fixation structure may take any of a variety of forms, such as one or more shafts, hooks, barbs, screws, sutures, clips, pincers, staples, tacks, or other fasteners. In some embodiments, the fixation structure can at least partially penetrate the mucosal lining of the urethral tract 17. In other embodiments, the fixation structure pinches or otherwise holds a fold of mucosal lining tissue. Alternatively, the fixation structure may take the form of an expandable frame attached to the housing of monitor 18. The expandable frame, as described in greater detail below, expands radially outward to engage the walls of urethra 16 and thereby secure monitor 18 in place at a desired position within urinary tract 17. Other possible fixation mechanisms include suction devices, magnetic devices, or helical screw-like mechanisms. In each case, the fixation structure securely maintains monitor 10 at a target location.

Examples of suitable biocompatible materials for fabrication of the fixation structure include stainless steel, titanium, polyethylene, nylon, PTFE, nitinol, or the like. In some embodiments, the fixation structure may be made form a degradable material that degrades or absorbs over time at the attachment site to release monitor 18 from tissue at the target location. In either case, upon detachment, sensor 18 can be recovered from urinary tract 17 of patient 12. U.S. Pat. Nos. 6,285,897 and 6,698,056 to Kilcoyne et al. provide examples of fixation mechanisms for attaching monitoring devices to the lining of the esophagus, including suitable degradable materials. The fixation structures described in the Kilcoyne et al. patents may be suitable for attachment of monitor 18. The contents of the Kilcoyne et al. patents are incorporated herein by reference in their entireties.

Examples of suitable degradable materials for fabrication of the fixation structure or structures include bioabsorbable or dissolvable materials such as polylactic acid (PLA) or copolymers of PLA and glycolic acid, or polymers of p-dioxanone and 1,4-dioxepan-2-one, as described in the Kilcoyne patents. A variety of absorbable polyesters of hydroxycarboxylic acids may be used, such as polylactide, polyglycolide, and copolymers of lactide and glycolide, as also described in the Kilcoyne patents. Other examples of degradable materials include polyether ketone (PEEK), carbohydrates or fibrin.

Alternatively, the fixation structure may include or take the form of a bonding agent such as a surgical adhesive that supplements the attachment made by the fixation mechanism or serves as the fixation mechanism itself. In other words, a pin, hook or other fixation mechanism may be accompanied by a bonding agent such as a biocompatible, surgical adhesive, or the adhesive may be used as the sole fixation structure without mechanical fasteners. Hence, the bonding agent may work alone or in combination with a mechanical fastener.

Examples of suitable boding agents for bonding monitor 18 to the mucosal lining include surgical adhesive such as any of a variety of cyanoacrylates, derivatives of cyanoacrylates, or any other adhesive compound with acceptable toxicity to human gastrointestinal cells that provides the necessary adhesion properties required to secure monitor 18 to the target location for a period of time sufficient for monitoring or delivery of electrical stimulation. Adhesives may be injected or otherwise applied into the region surrounding the target location, e.g., via a channel within the endoscopic delivery device, or carried by the monitor 18 itself.

Other examples of suitable bonding agents include biologically medicated bonding agents such as fibrin glues. Fibrin glue is a biological tissue adhesive found to be an effective sealant and topical hemostatic agent. An example of a commercially available fibrin glue is marketed as Tissucol. Fibrin glue generally includes concentrated fibrinogen and factor XII combined with thrombin and calcium to form a coagulum. This preparation stimulates the final stage of the clotting cascade, producing a fibrin clot from fibrinogen in the presence of calcium within seconds after administration of the thrombin-activating solution. Other biologically mediated bonding agents that may be suitable include glues based on collagen, albumin or gelatin.

As further shown in FIG. 1, in some embodiments, monitor 18 may communicate with an external receiver 20 via wireless telemetry. External receiver 20 may permit a user to retrieve physiological information obtained by a sensor carried by monitor 18. In addition, as will be described, external receiver 20 may process information obtained from the sensor, and present the information to a user via a display or other output media. The information may include one or more advisories with respect to the presence or level of a urodynamic parameter or urine physical characteristic. External receiver 20 may present recommendations for delivery or modification of therapy, such as intake of pharmaceuticals, based on the information. In addition, in some embodiments, external receiver 20 may automatically adjust therapies applied by devices such as neurostimulators or drug pumps.

Wireless telemetry may be accomplished by radio frequency communication or proximal inductive interaction of external receiver 20 with monitor 18. In some embodiments, telemetry for purposes of controlling the detachment mechanism may be accomplished by simply passing a magnet over monitor 18 or inductively powering the medical device via an inductive coil interface. External receiver 20 may take the form of a portable, handheld device, like a pager or cell phone, that can be carried by patient 12. External receiver 20 may include an antenna that is attached to the body of patient 12 at a location proximate to the location of monitor 18 to improve wireless communication reliability. Also, in some embodiments, external receiver 20 may receive operational or status information from monitor 18, and may be configured to actively interrogate the medical device to receive the information.

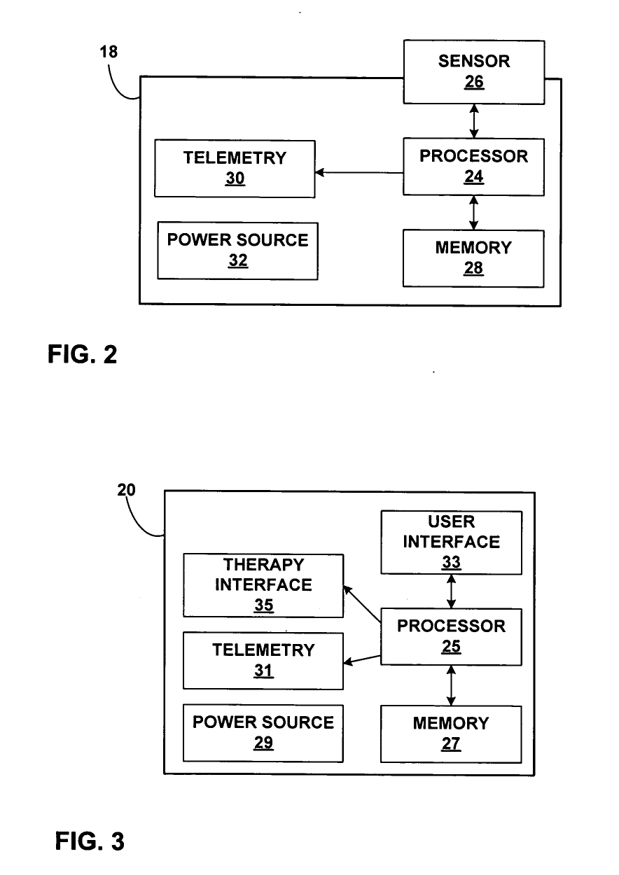

FIG. 2 is a block diagram illustrating exemplary functional components of a urethral tract monitor 18. In the example of FIG. 2, monitor 18 may include a processor 24, a sensory 26, memory 28, telemetry unit 30, and a power source 32. Power source 32 may take the form of a small battery. In some embodiments, medical device 20 may further include an inductive power interface to power some functions of monitor 18, such as telemetry. Telemetry unit 30 permits communication with external receiver 20 for transfer of information. In some embodiments, however, telemetry module 30 may be optional. For example, monitor 18 may exclude telemetry module 30 if data is to be stored in memory 28, and then acquired from the monitor after retrieval from urinary tract 17. Exclusion of telemetry unit 30 may be desirable in some applications to achieve reductions in the size of monitor 18.

Processor 24 controls operation of monitor 18 and may include one or more microprocessors, digital signal processors (DSPs), application-specific integrated circuits (ASICs), field-programmable gate arrays (FPGAs), or other equivalent logic circuitry. Memory 28 may include any magnetic, electronic, or optical media, such as random access memory (RAM), read-only memory (ROM), electronically-erasable programmable ROM (EEPROM), flash memory, or the like. Memory 28 may store program instructions that, when executed by processor 24, cause the controller to perform the functions ascribed to it herein. For example, memory 28 may store instructions for processor 24 to execute in support of control of telemetry unit 30 and sensor 26.

Telemetry unit 30 may include a transmitter and receiver to permit bi-directional communication between monitor 18 and external receiver 20. In this manner, external receiver 20 may transmit commands to monitor 18 and receive status and operational information from the monitor. Telemetry unit 30 includes an antenna, which may take a variety of forms. For example, the antenna may be formed by a conductive coil or wire embedded in a housing associated with monitor 18. Alternatively, the antenna may be mounted on a circuit board carrying other components of monitor 18, or take the form of a circuit trace on the circuit board. If monitor 18 does not include a telemetry unit 30, a magnetic reed switch may be provided in a circuit so that monitor 18, with the aid of an external magnet, may activate or deactivate itself in response to external input.

Battery power source 32 may take the form of a battery and power circuitry. Monitor 18 typically may be used for a few days or weeks, and therefore may not require substantial battery resources. Accordingly, the battery within battery power source 32 may be very small. An example of a suitable battery is the Energizer 337 silver oxide cell, available from the Eveready Battery Company, of St. Louis, Mo., USA. The Energizer 337 battery is disc-shaped, and has a diameter of 4.88 mm and thickness of 1.65 mm. With a typical range of power requirements for sensing applications, this battery can be expected to power monitor 18 for between approximately forty-eight hours and twenty days, depending on actual usage conditions. Another example battery is the QL003I 3 milliamp cylindrical battery from Quallion, LLC, of Sylmar, Calif., USA, which has a diameter of approximately 2.9 mm and a length of approximately 13.0 mm. The Quallion battery is rechargeable and could last several months with periodic recharging, e.g., by inductive charging circuitry. The sample rate and type of sensor used will determine battery longevity. Sample rates may vary from once per day to 100 Hz, depending on the monitoring application. As examples, a pH sensor may take samples every 6 seconds, whereas a pressure sensor adapted to sense pressure during a urine voiding event, may sample at rates up to or exceeding 100 Hz.

Different types of batteries or different battery sizes may be used, depending on the requirements of a given application. In further embodiments, battery power source 32 may be rechargeable via induction or ultrasonic energy transmission, and includes an appropriate circuit for recovering transcutaneously received energy. For example, battery power source 32 may include a secondary coil and a rectifier circuit for inductive energy transfer. In still other embodiments, battery power source 32 may not include any storage element, and monitor 18 may be fully powered via transcutaneous inductive energy transfer.

Sensor 26 may be selected for any of a variety of urodynamic testing applications or urinalysis applications, and may include appropriate signal processing circuitry such as amplifier, filter, and analog-to-digital conversion circuitry for presentation of sensed information to processor 24. For urodynamic testing, sensor 26 may take the form of a pressure, flow, volume, or temperature sensor. In some embodiments, pressure or other measurements may be used to detect bladder or urinary sphincter functions. For urinalysis, sensor 26 may be configured to detect a variety of physical characteristics of urine such as pH, temperature, odor, color, or the like. In addition, sensor 26 may target the presence or levels of specific physical characteristics such as urine acidity or the presence of sugar, proteins, blood, keytones, bilirubin or other substances such as bacteria, yeast cells or parasites in the urine. Further, sensor 26 may be configured to detect the presence of drug residue in the urine, such as the presence of alcohol, or the presence of drugs such as marijuana, cocaine, heroin, or other controlled substances.

FIG. 3 is a functional block diagram illustrating an external receiver 20 for communication with urinary tract monitor 18 of FIG. 2. In the example of FIG. 3, external receiver includes a processor 25, memory 27, power source 29, telemetry unit 31, user interface 33, and optionally a therapy interface 35. Memory 27 stores instructions for execution by processor 25. In addition, memory 27 may store information received from monitor 18 over a period of observation, thereby reducing the memory requirements, and hence size and power consumption, of the monitor. Processor 25 controls telemetry interface 29 to obtain information from monitor 18, and presents information to a user via user interface 33. User interface 33 may include a display or other visual media for presentation of information, and may further include audible media for presentation of audible tones, speech messages, or other audio information. The information presented via user interface 33 is based on information obtained from monitor 18, and may include advisories, sensed levels, indications of detected substances, and the like. The user may be patient 12 or a physician, nurse or other health care worker or care provider.

Processor 29 may control telemetry unit 29 to receive information from monitor 18 on a substantially continuous basis, at periodic intervals, or upon user command. Hence, external receiver 20 may provide on ongoing, up-to-date indication of the physiological conditions sensed by monitor 18. In this manner, monitor 18 and external receiver 20 provide a convenient way to track the present status of conditions within urinary tract 17, permitting generation of historical data, trend data, and even instantaneous advisories in the event a sensed condition does not satisfy a desired threshold. As an example, if the sensed condition is glucose level, monitor 18 may provide a continuous, periodic or on-demand indication of the glucose level and generate an advisory via external receiver 20 in the event the level is too high or too low. This feature may enable a diabetic patient 12 to seek medical attention or self-administer a dose of insulin to moderate the glucose level. In some embodiments, external receiver 20 may generate a control signal to automatically adjust a therapy, such as an insulin dosage administered by an implantable or external insulin pump.

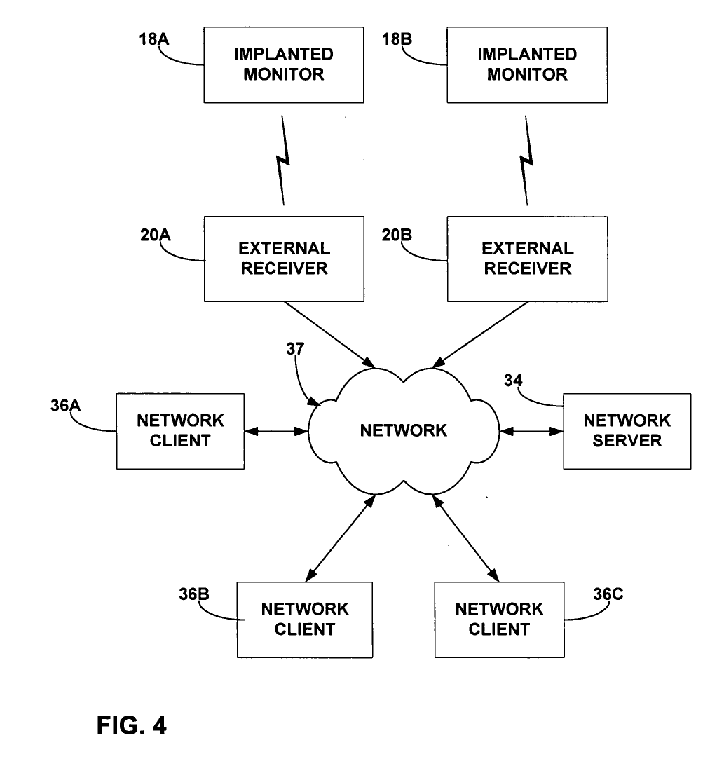

FIG. 4 is a functional block diagram illustrating a network 37 for communication of information obtained by one or more urinal tract monitors 18. Two implanted urinary tract monitors 18A, 18B are shown for purposes of illustration. However, information for any number of monitors 18 and patients 12 may be accessed via network 37. In particular, physicians or other medical personnel may view information transmitted to external receivers 20A, 20B by implanted monitors 18A, 18B to evaluate urodynamic conditions or urinalysis results. External receivers 20A, 20B are coupled to network 37 via wired or wireless connections, and transmit information obtained from monitors 18A, 18B to a network server 34 via the network.

Network server 34 may be equipped to analyze the information and generate appropriate reports or advisories for viewing by users via any of network clients 36A, 36B, 36C (collectively 36), coupled to network 37. For example, network server 34 may generate web pages or other output that conveys information obtained by monitors 18A, 18B. Hence, network clients 36 may access information on network server 34 using web browsers. In this manner, one or more users, such as physicians, may remotely view the results of urodynamic testing or urinalysis. Network 37 may take the form of a local area, wide area or global computer network, such as the Internet.

The information sent by external receivers 20A, 20B may be updated on a continuous or periodic basis, and even provide near real-time updates in some embodiments. Network server 34 may present urodynamic test results, urinalysis results, levels of particular parameters or physical characteristics, and recommended treatments, therapies or dosages based on the information. In some embodiments, network server 34 may be configured to poll external receivers 20 to received information. Network server 34 also may be configured to transmit advisories by email, facsimile, text messaging, instant messaging or the like to network clients 36, particularly for urinalysis results.

For example, if a patient's glucose level is at a level indicating an imminent health risk, as indicated by information transmitted by a monitor 18 to external receiver 20, network server 34 may respond by sending an advisory to a physician or other health care personnel so that medical attention can be provided immediately. As another example, if urinalysis performed by monitor 18 indicates ingestion of an illegal drug, e.g., in violation of a felon's parole conditions, network sever 34 may transmit an advisory to a law enforcement agent via one of network clients 36. In either case, the user associated with a network client 36 is able to remotely monitor information concerning a patient's condition, as obtained by the implanted monitor 18, and act on that information, if appropriate.

The ability to perform urinanalysis or urodynamic testing with a temporary or chronic implanted monitor 18, combined with remote monitoring capabilities, can support a wide range of patient management capabilities, tight control of drug management, disease diagnostics, and chronic disease management. In addition, the ability to perform urinanalysis or urodynamic testing while that patient is at home and going about daily living activities can provide much more accurate and meaningful data. For example, a pressure monitor in the bladder may be used to assess bladder function over a period of several days, and over the course of several activities such as rest, eating, drinking, and exercise.

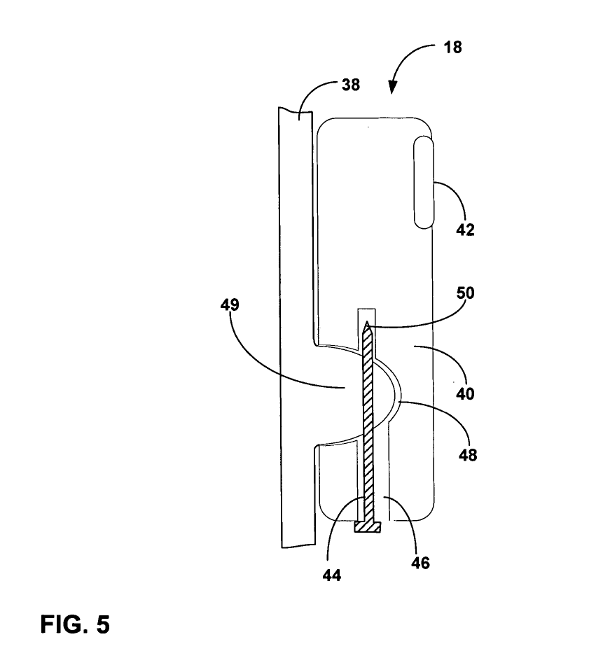

FIG. 5 is a cross-sectional side view of a urinary tract monitor 18 with a fixation structure in accordance with an embodiment of the invention. In the example of FIG. 5, monitor 18 is placed adjacent mucosal lining 38 within bladder 14 or urethra 16. Monitor 18 includes a capsule-like housing 40. A sensor 42 is exposed by housing 40 for interaction with the environment within bladder 14 or urethra 16. A shaft 44 extends through an internal channel 46 in the capsule-like housing of monitor 18. Monitor 18 defines a vacuum cavity 48 on a side of the housing adjacent mucosal lining 38. A vacuum port defined by channel 46 applies vacuum pressure to vacuum cavity 48 to draw a portion of mucosal tissue 49 into the cavity. The vacuum port is attached to a vacuum line (not shown) carried by an endoscopic delivery device. The vacuum line is coupled to an external vacuum source.

An elongated control rod (not shown in FIG. 5) may be applied via the endoscopic delivery device to drive shaft 48 into mucosal tissue 49. Shaft 44 has a sharpened tip 50 that facilitates partial or complete penetration of tissue 49. Upon penetration of tissue 49 to secure monitor 18 relative to mucosal lining 38, vacuum pressure is deactivated and the endoscopic delivery device is withdrawn from urethra 16. Although shaft 44 is illustrated as penetrating tissue 49, in some embodiments, the shaft may be spring-biased to pinch a fold of the tissue and thereby secure monitor 18 at a desired position.

As discussed above, shaft 44 may be manufactured from degradable materials that degrade over time, e.g., in the presence of urine, to release monitor 18 from mucosal lining 38. Alternatively, monitor 18 may release from mucosal lining 38 as mucosal tissue 49 sloughs away from mucosal lining 38. In either case, once the mucosal tissue 49 is released by shaft 44, monitor 18 detaches from mucosal lining 38 for passage through the urinary tract with urine flow or recovery with an endoscopic recovery device. In general, shaft 44, vacuum cavity 48 and the vacuum port defined by channel 46 form a fixation structure. In general, monitor 18 may make use of fixation structures that are configured and function in a manner similar to any of the fixation structures disclosed in the above-referenced Kilcoyne patents.

Sensor 42 is selected to sense one or more physiological conditions within urinary tract 17. The physiological conditions may be urodynamic parameters or physical characteristics of urine. The information obtained by sensor 42 may be used to diagnose a variety of conditions or disorders. As examples, for urodynamic testing, sensor 42 may sense urine pressure, flow, velocity, or urine volume within bladder 14. Sensor 42 may have a structure similar to sensors conventionally used for catheter-based urodynamic testing. For pressure measurements, for example, sensor 42 may include one or more diaphragm sensors, strain gauge sensors, capacitive sensors, piezoelectric sensors, or other sensors used in conventional catheter-based urodynamic testing to sense pressure. For bladder emptying, sensor 42 may include a conductive sensor to sense the presence of urine within the lower region of the bladder 14.

For flow measurements, sensor 42 may comprise a pulsed Doppler ultrasonic sensor, or a laser Doppler flow sensor. Doppler shifting of the frequency of the reflected energy indicates the velocity of the fluid flow passing over a surface of sensor 42. Consequently, in some embodiments, monitor 18 may include circuitry, such as a quadrature phase detector, in order to enable the monitor to distinguish the direction of the flow of fluid in addition to its velocity. As a further example, sensor 42 may include any one or more thermal-convection velocity sensors. A thermal-convection velocity sensor may include a heating element upstream of the thermistor to heat urine within the urethra 16 such that flow rate may be measured according to the temperature of the heated fluid when it arrives at the thermistor. In other embodiments, flow rate may be determined from the output of a concentration or temperature sensor using Fick's techniques.

By monitoring pressure over a period of time, monitor 18 can provide information indicative of frequency or urination and amount of pressure the bladder 14 is able to produce. With information about fluid flow rate, bladder pressure, and timing of voiding, monitor 18 may serve as a useful diagnostic tool for many disorders, such as BPH. Also, obtaining urodynamic information over a period of several days and in a patient's home environment may be particularly useful. For example, the urodynamic results during the night may be different from the results during the daytime.

For urinalysis, a multitude of different sensor types may be used for sensor 42. Conventional pH sensors, temperature sensors, or other sensors may be used. Urine color may be determined by analyzing optical parameters. In addition, a concentration of ions or other solutes present in body fluids can be detected and analyzed using sensor 42, e.g., by electrochemical sensing. For example, a sensor 42 capable of sensing ions such as sodium, potassium, calcium, magnesium, chloride, bicarbonate, or phosphate may be incorporated in monitor 18. Sensor 42 may be configured to sense other solutes with concentrations of interest, such as glucose, bilirubin, creatinine, blood urea nitrogen, leukocyte esterase, urobilinogen, urinary nitrogen, renin, and angiotensin. In addition, sensor 42 may detect other substances such as illegal drugs, alcohol, sugar, proteins, blood, keytones, bilirubin or even bacteria, yeast cells or parasites in the urine.

Although sensor 42 is depicted as having one or more surface components exposed to an environment within bladder 14 or urethra 16, in some embodiments, monitor 18 may include a hollow lumen to allow urine flow through the monitor. In this case, monitor 18 may have an annular cross-section, in a plane perpendicular to urine flow, and sensor 42 may be oriented such that sensor components are exposed to the interior of the hollow lumen. This type of configuration for monitor 18 may be particularly useful within urethra 16, and can be used to monitor flow rate, pressure, and timing of voiding, which may be advantageous in diagnosing BPH.

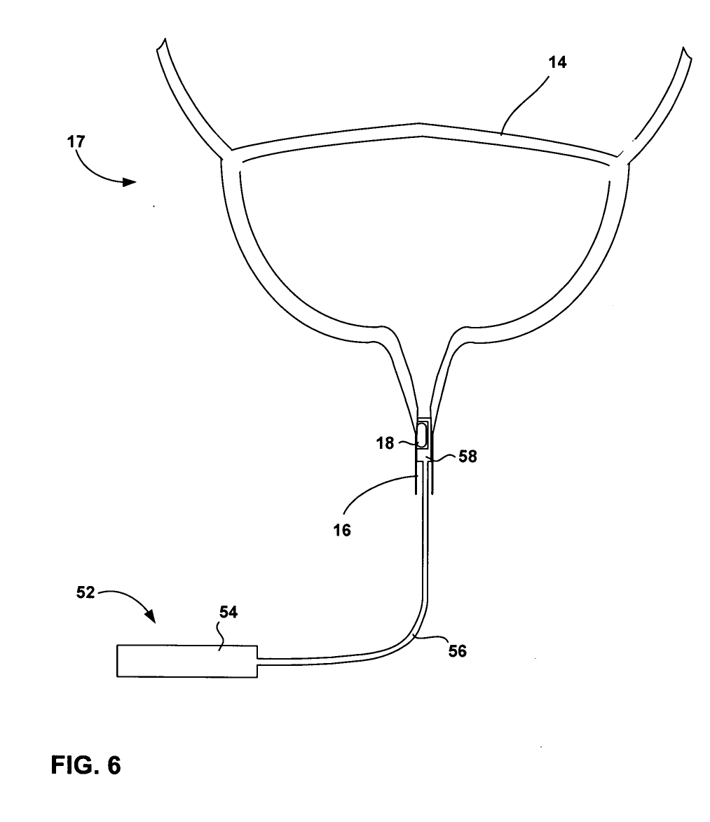

FIG. 6 is a schematic diagram illustrating deployment of a monitor 18 within a patient's urinary tract 17. As shown in FIG. 6, an endoscopic delivery device 52 serves to position and place monitor 18 within urinary tract 17 of patient 12. Delivery device 52 includes a proximal portion, referred to herein as a handle 54, and a flexible probe 56 that extends from handle 54 for insertion into urethra 16. Probe 56 is sized for passage through urethra 16 and may include a lubricating coating to facilitate passage.

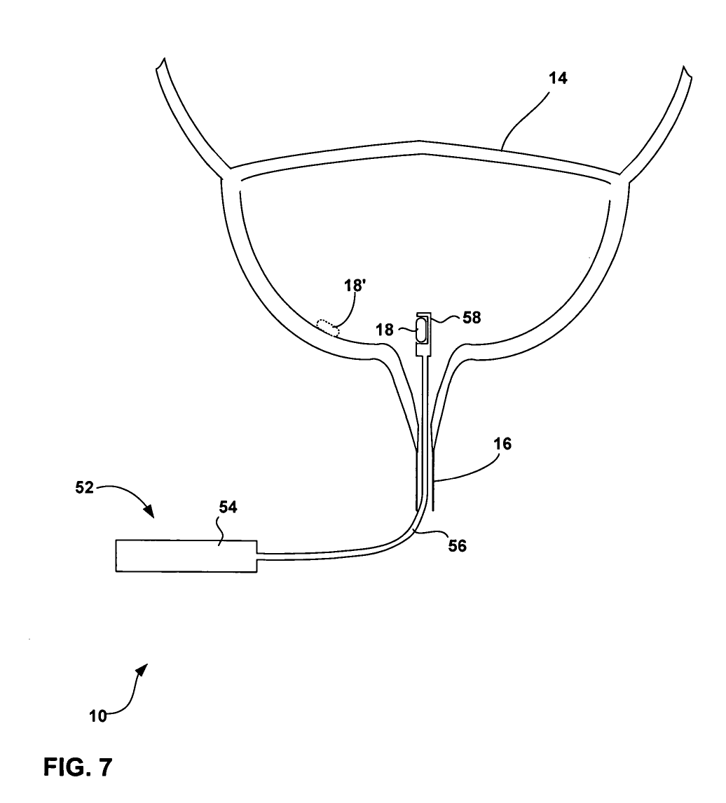

Monitor 18 is coupled to a distal end 58 of delivery device 52 for delivery to a target location within the urinary tract 17. The target location may be within urethra 16 or within bladder 14. FIG. 7 a schematic diagram illustrating further deployment of monitor 18 within bladder 14 using endoscopic delivery device 52. In some embodiments, delivery device 52 may include appropriate guidewires or other steering mechanisms to permit placement of monitor 18 on a lateral wall of bladder 14, as indicated by the position of monitor 18′.

Distal end 58 of delivery device 52 enters urethra 16 and extends into the urethra to the target location. The progress of distal end 58 may be monitored by endoscopic viewing or external viewing, e.g., with ultrasound or fluoroscopy. Monitor 18 is attached to the mucosal lining at the target location within bladder 14 or urethra 16, and the distal end 58 of delivery device 52 releases the monitor. Upon placement of monitor 18, flexible probe 56 and distal end 58 are withdrawn from urethra 16. Monitor 18 may be activated prior to placement within urinary tract 17, or activated remotely by wireless communication or passage of a magnetic in close proximity to monitor 18 to activate a switch carried by the monitor.

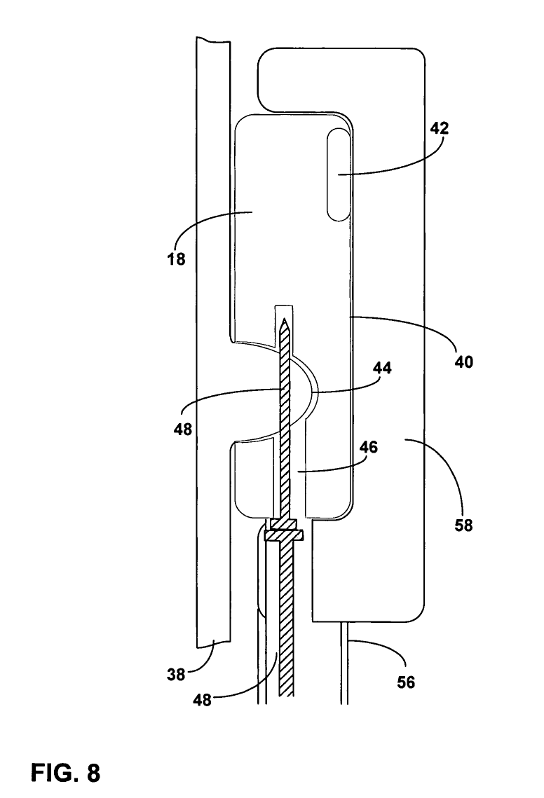

FIG. 8 is a cross-sectional side view illustrating positioning of monitor 18 of FIG. 4 within distal end 58 of an endoscopic delivery device 52. As shown in FIG. 8, monitor 18 is held within a placement bay 57 within distal end 58 of endoscopic delivery device 52. In this example, a physician advances elongated control rod 59 to drive shaft 48 into mucosal tissue 49. In general, elongated control rod 59 permits a physician to exert force to penetrate mucosal tissue 49. Elongated control rod 59 is flexible and extends though flexible probe 56 to handle 54 so that the physician can manipulate the elongated control rod. Before advancing elongated control rod 59, however, the physician activates a vacuum line to supply vacuum pressure to vacuum cavity 48 via channel 46 of monitor 18.

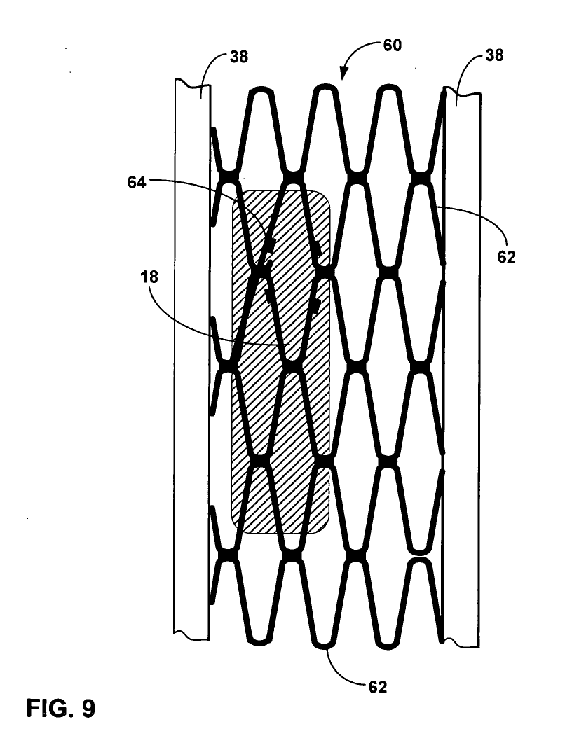

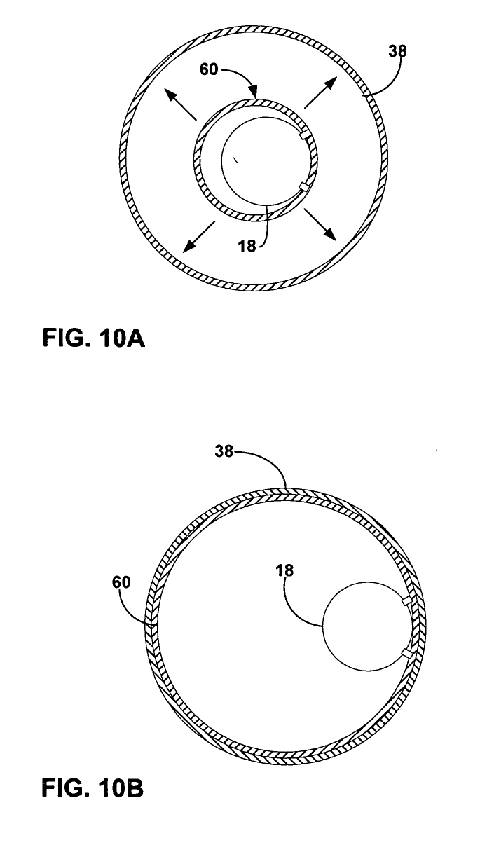

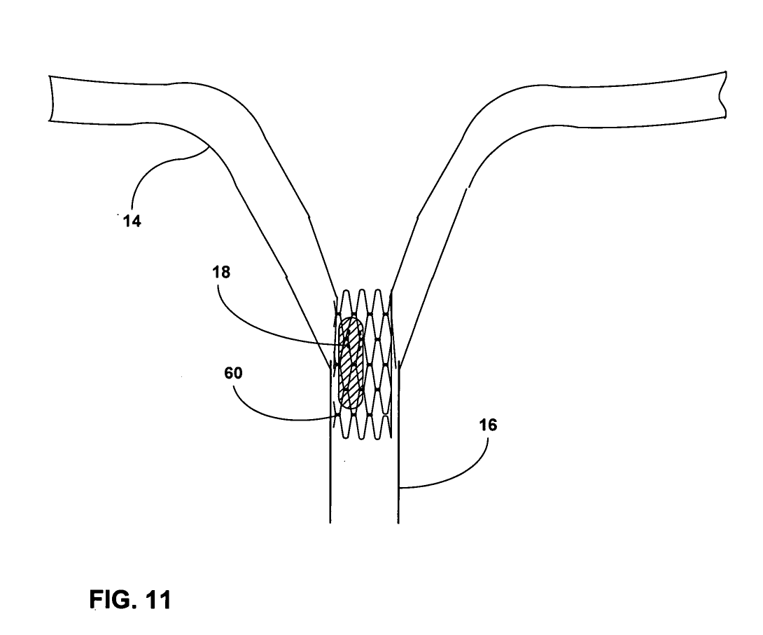

FIG. 9 is a side view of a monitor 18 with another fixation structure in the form of an expandable frame 60. FIG. 10A is a cross-sectional end view of the monitor 18 and expandable frame 60 of FIG. 9 in an unexpanded state within the urethra 16. FIG. 10B is a cross-sectional end view of the monitor 18 and expandable frame 60 of FIG. 9 in an expanded state within the urethra 16. As shown in FIGS. 9, 10A, and 10B, the capsule-like housing of monitor 18 has a diameter that is substantially less than the diameter of expandable frame 60 when the frame is in a fully expanded state. Upon expansion, frame 60 engages the mucosal lining of the interior wall of urethra 16, much like a conventional stent used for restoring patency of blood vessels. In this manner, expandable frame 60 securely holds monitor 18 in place at a target location within the urethra 16. FIG. 11 is another side view of the monitor 18 and expandable frame 60 of FIG. 9 positioned within the urethra 16.

As shown in FIGS. 9-11, the capsule-like housing of monitor 18 is attached to a portion of a wire grid 62 forming expandable frame 60. Monitor 18 may be welded, adhesively bonded, or crimped to one or more coupling points 64 on expandable frame 60. Wire grid 62 may take the form of a grid, network, or mesh of elastic wires that form a substantially cylindrical frame, similar to a conventional stent useful in restoring blood vessel patency. Examples of suitable materials for fabrication of wire grid 62 include stainless steel, titanium, nitinol, and polymeric filament, which can be absorbable or nonabsorbable in vivo, as described in the above-referenced Kilcoyne patents.

Expandable frame 60 may be intrinsically elastic such that it is self-expandable upon release from a restraint provided by an endoscopic delivery device. Alternatively, in some embodiments, a balloon or other actuation mechanism may be used to actively expand frame 60 to a desired diameter. In each case, as shown in FIGS. 10A and 10B, expandable frame 60 extends radially outward to engage the wall of a urethra 16, and thereby place monitor 18 in contact with the lumen wall. In particular, upon expansion of frame 60, monitor 18 is placed within the lumen defined by urethra 16, and within the flow of urine through the urethra.

The position of monitor 18 within urethra 16 permits sensing of urodynamic parameters, such as pressure, flow rate, temperature, and the like. In addition, monitor 18 is in contact with urine flow to sense any of a variety of physical characteristics for urinalysis. Monitor 18 senses the applicable physiological conditions and transmits information based on the sensed conditions to external receiver 20. In some embodiments, expandable frame 60 may be electrically coupled to monitor 18 and form part of an antenna to facilitate reliable wireless telemetry.

Monitor 18 is depicted in FIGS. 9-11 as being coupled to one side of expandable frame 60, and therefore resides adjacent a wall of urethra 16. In other embodiments, however, monitor 18 may be mounted to frame 60 such that monitor resides substantially centrally within urethra 16. For example, monitor 18 may be cantilevered or otherwise supported by frame 16 with expandable struts that place the monitor centrally within the aperture defined by the frame. In this case, monitor 18 may be constructed with a hollow lumen for passage of urine flow, and a sensor associated with monitor 18 may be oriented inward toward the lumen to sense conditions of the urine such as urodynamic conditions or urinalysis characteristics.

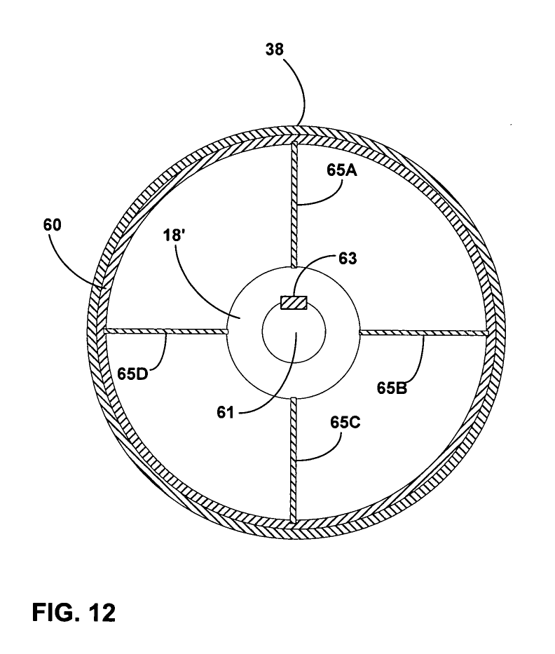

FIG. 12 is a cross-sectional view of an alternative monitor 18′ mounted to an expandable frame 60. Monitor 18′ includes central lumen 61 and a sensor 63 mounted to face inward into lumen 61. In the example of FIG. 12, monitor 18 is centrally mounted within frame 60 via struts 65A, 65B, 65C, 65D. Monitor 18′ and frame 60 may be mounted within urethra 16 such that sensor 63 monitors flow rate, pressure or other urodynamic parameters associated with urine passing through lumen 61. Alternatively, sensor 63 may be configured for urinalysis of urine passing through lumen 61. In either case, urine is free to flow through central lumen 61 of monitor 18′, and around the monitor through expandable frame 60. Monitor 18′ may be placed within urethra 16 downstream from the prostate gland, and be particularly useful in detecting BPH or other restrictive disorders of the urethra.

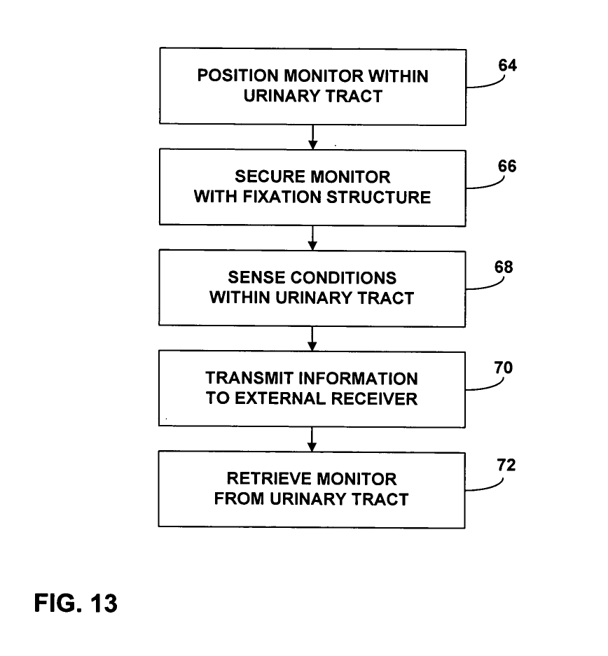

FIG. 13 is flow diagram illustrating a method for placement and use of an implantable urinary tract monitor 18 in accordance with the invention. In the example of FIG. 13, the method involves positioning a monitor within the urinary tract 17 using an endoscopic delivery device (64), and securing the monitor at a target location with a fixation structure (66). Again, the fixation structure may include a vacuum cavity and shaft to penetrate or pinch captured tissue, an expandable stent-like frame, or other structures for attaching monitor 18 to urethral or bladder tissue or otherwise maintaining the monitor at a particular position.

As further shown in FIG. 13, following placement, the monitor 18 senses conditions within the urinary tract (68), such as urodynamic parameters, physical urine characteristics or both, and transmits the information based on the sensed conditions to external receiver 20 (70). Finally, when a sufficient amount of information has been obtained, a physician retrieves monitor 18 from the urinary tract 17 (72).

The physician may use an endoscopic retrieval device such as a surgical snare, jaws, or the like. Alternatively, in some embodiments, monitor 18 may release from tissue within the urinary tract 17 due to degradation of the fixation structure or sloughing of tissue to which the monitor is attached. In this case, monitor 18 may be retrieved or possibly pass from urinary tract 17 with urine flow.

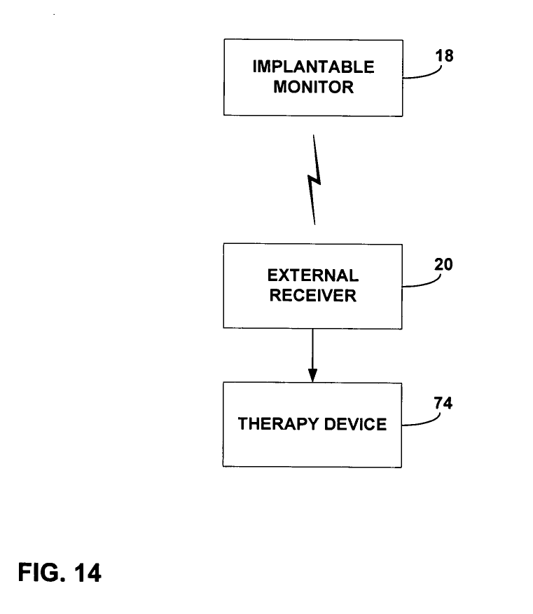

FIG. 14 is a functional block diagram illustrating communication of information from an implantable urinary tract monitor 18 to an external receiver 20 to control a therapy device 74. As previously described with respect to FIG. 3, external receiver 20 may include a therapy interface that permits the external receiver to control or adjust a therapy applied to patient 12 by an implanted or external therapy device. External receiver 20 generates a control signal to adjust the therapy based on information received from implanted monitor 18. In this manner, external receiver 20 can be configured to take advantage of continuous, periodic, on-demand monitoring of physiological conditions within the urinary tract 17 by monitor 18. In response to information received from monitor 18, a processor within external receiver 20 analyzes current conditions, e.g., by comparing parametric levels to applicable thresholds, to determine an adjustment to a therapy such as neurostimulation or drug delivery.

As one example, if monitor 18 indicates that a glucose level is too high or too low, external receiver 20 may generate a control signal and transmit the control signal to an external or implanted insulin pump to administer or adjust a dosage of insulin and thereby moderate the glucose level to a desired range. As another example, external receiver 20 may respond to a urodynamic measurement that indicates insufficient urine flow or emptying of the bladder. In particular, external receiver 20 may transmit a control signal to an implanted neurostimulator to apply electrical stimulation to bladder or urinary sphincter muscles to improve urodynamic function.

External receiver 20 transmits the control signal to therapy device 74 by wired or wireless communication. In some embodiments, it is conceivable that monitor 18 may be configured to generate a control signal for transmission to therapy device 74. Typically, however, external receiver 20 will be equipped to analyze information transmitted by monitor 18 to generate control signals. With continuous, periodic or on-demand monitoring by monitor 18, external receiver 20 supports a closed-loop feedback system that is responsive to actual conditions within the urinary tract of patient 12 at a given time. External receiver 20 and monitor 18 can be used in this manner to provide therapies selected to support improved urinary function.

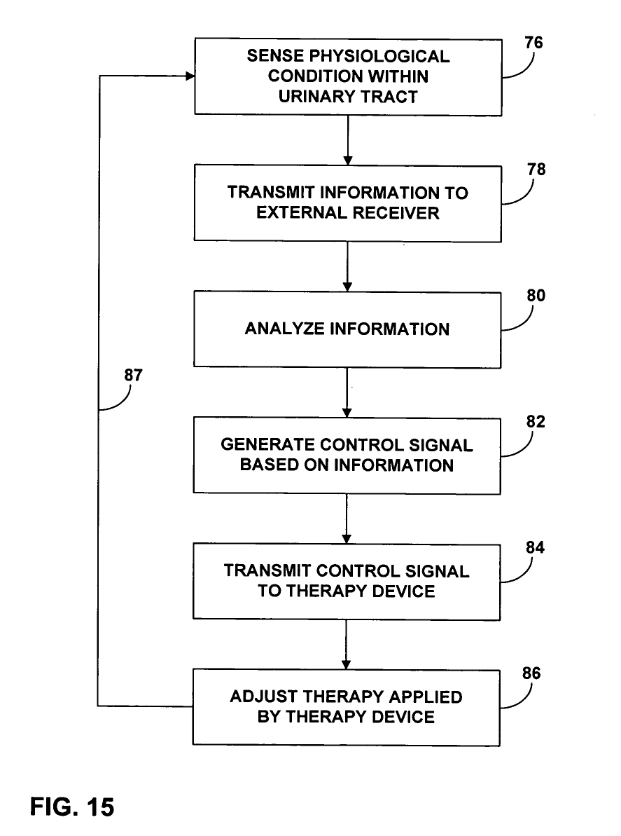

FIG. 15 is a flow diagram illustrating communication of information from an implantable urinary tract monitor 18 to an external receiver 20 to control a therapy device 74. As shown in FIG. 15, monitor 18 senses a physiological parameter of urinary tract 17 (76), and transmits information based on the physiological condition to external receiver 20 (78). External receiver 20 analyzes the information received from monitor 18 (80), and generates a control signal based on the information (82). External receiver 20 then transmits the control signal to a therapy device 74 (84) to adjust a therapy applied by the therapy device (86). This process of receiving and analyzing the information from monitor 18 may be performed continuously, periodically or on an on-demand basis, as represented by loop 87.

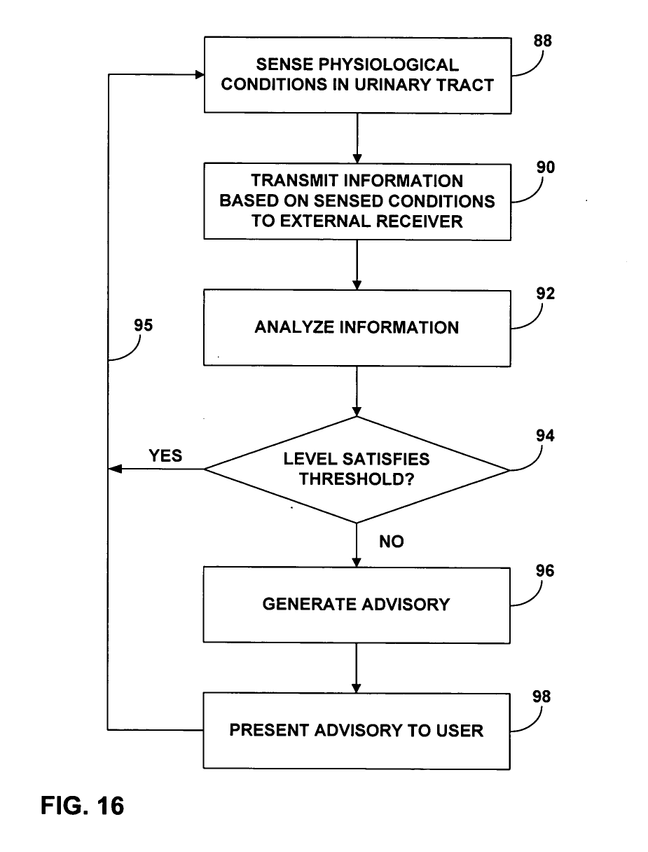

FIG. 16 is a flow diagram illustrating communication of information from an implantable urinary tract monitor 18 to an external receiver 20 to generate advisories. As an alternative or in addition to automated control of therapy devices, external receiver 20 may generate advisories in response to information received from monitor 18. The advisories may be presented via a user interface associated with external receiver 20. For example, a user interface associated with external receiver 20, as described with respect to FIG. 3, may include a display or other visual media for presentation of advisories, as well as audible media for presentation of audible tones, speech messages, or other audio information to convey advisories.

As shown in FIG. 16, monitor 18 senses a physiological condition within urinary tract 17 (88), and transmits information based on the sensed condition to external receiver 20 (90). External receiver 20 then analyzes the information received from monitor 18 (92). For example, external receiver 20 may compare a level of physiological condition, such as a urodynamic parameter or physical urine characteristic, to a threshold or range. If the level satisfies the threshold or range (94), external receiver 20 does not generate an advisory and the process continues as indicated by loop 95. If the physiological condition does not satisfy the threshold or range (94), however, external receiver 20 generates an advisory (96), and presents the advisory to a user (98).

Again, external receiver 20 may present the advisory via a user interface associated with the receiver. In this case, the advisory may be observed by a patient or other user in possession of external receiver 20. Alternatively, external receiver 20 may transmit the advisory to a different device. As an illustration, external receiver 20 may transmit the advisory to a network server, as depicted in FIG. 4, so that one or more users may remotely receive the advisory. In each case, a user may take action in response to the advisory, such as providing, recommending or scheduling a medical examination or therapy. In some instances, the advisory may represent a condition that requires immediate medical attention, and may promote the patient or a physician to pursue the medical attention.

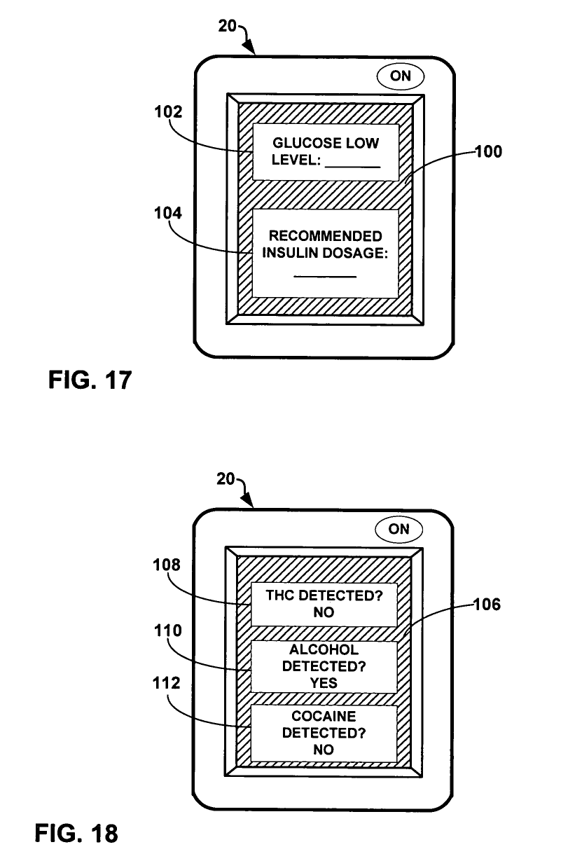

FIG. 17 is a conceptual diagram of an external receiver 20 equipped to communicate an advisory with respect to a sensed condition. In the example of FIG. 17, external receiver 20 includes a display screen 100 that presents two advisories 102, 104. Advisory 102 indicates that the patient's glucose level is low, and may indicate the actual level of glucose. In this case, external receiver 20 analyzes information received from implanted monitor 18 to determine whether the glucose level falls within a particular range. If not, external receiver 20 indicates whether the glucose level is low or high. In addition, external receiver 20 presents advisory 104, which recommends an insulin dosage to moderate the glucose level toward the desired range.

FIG. 18 is a conceptual diagram of another external receiver 20 equipped to communicate an advisory with respect to a sensed condition. In the example of FIG. 18, external receiver 20 includes a display that presents multiple advisories with respect to detection of particular substances, such as drug residue, within the patient's urine. In this case, monitor 18 performs one or more urinalysis routines to sense the presence of drug residue. If particular substances are detected, external receiver 20 presents advisories 108, 110, 112 which, in this example, indicate the presence or absence of THC, alcohol or cocaine in the patient's urine.

While FIG. 17 illustrates an embodiment in which external receiver 20 presents advisories helpful to a patient or physician, FIG. 18 illustrates the presentation of advisories that may be helpful to drug testing organization, employers, or law enforcement personnel. In each case, external receiver 20 relies on indwelling urinalysis results. In other examples, however, external receiver 20 may present advisories, test results or other information conveying urodynamic testing results for use by a physician in evaluating urinary tract function and prescribing appropriate therapy for a patient. In addition, external receiver 20 may be configured to transmit control signals to other devices to provide biofeedback in response to sensed conditions in urethral tract 17. Alternatively, the advisories generated by external receiver 20 also may serve as biofeedback to take steps to modify the patient's behavior to alleviate symptoms of urinary tract disorders. If monitor 18 generates pressure information indicative of bladder fullness or an imminent need to void urine, for example, external receiver 20 may generate an advisory that prompts the incontinent patient to take steps before the need becomes urgent.

The preceding specific embodiments are illustrative of the practice of the invention. It is to be understood, therefore, that other expedients known to those skilled in the art or disclosed herein may be employed without departing from the invention or the scope of the claims. For example, the invention is not limited to deployment of a monitor at a particular location within the urinary tract. In various embodiments, a medical device may be located anywhere within the urinary tract where useful diagnostic information can be obtained.

The invention also is not limited to monitoring particular physiological conditions. Instead, a monitor as described herein may be used for urodynamic testing, urinalysis, or other diagnostic evaluations pertinent to the urinary tract. In addition, for embodiments in which information obtained by the monitor may be used to control or adjust therapy devices, the therapies need not be limited only to neurostimulation or drug delivery, but may encompass other therapies useful in treating conditions or disorders within the urinary tract. Moreover, the invention is not limited to application for monitoring associated with any particular disorder, condition or affliction. As further examples, a monitoring device in accordance with the invention can be used to monitor other types of physiological conditions, such as conditions indicative of pregnancy, ovulation, or the condition of a fetus.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A monitor for placement within a urinary tract of a patient, the monitor comprising:
a monitor housing sized for introduction into a urethra of a patient;
an expandable frame mounted to the device housing to secure the device housing at a position within the urinary tract; and
a sensor to sense one or more physiological conditions within the urinary tract.

2. The monitor of claim 1, wherein the sensor is configured to sense one or more urodynamic parameters.

3. The monitor of clam 2, wherein the urodynamic parameters includes at least one of urine pressure, urine flow, urine pH, temperature, and bladder contraction.

4. The monitor of claim 1, wherein the sensor is configured to sense one or more physical characteristics of urine in the urinary tract.

5. The monitor of claim 4, wherein the physical characteristics include presence of drug residue in the urine.

6. The monitor of claim 4, wherein the physical characteristics include presence of at least one of sugar, proteins, blood, keytones, bilirubin, bacteria, yeast cells, and parasites in the urine.

7. The monitor of claim 4, wherein the physical characteristics include glucose level.

8. The monitor of claim 4, further comprising a telemetry unit to transmit signals indicative of the sensed conditions.

9. The monitor of claim 1, wherein the expandable frame includes a wire grid formed from one of stainless steel, titanium, nitinol, and polymeric filament.

10. The monitor of claim 1, wherein the expandable frame is elastic and self-expandable.

11. The monitor of claim 1, wherein the monitor housing is substantially cylindrical in shape, and defines a diameter substantially less than a fully expanded diameter of the expandable frame.

12. The monitor of claim 1, further comprising a power source to power the sensor over a period of at least twenty-four hours.

13. The monitor of claim 1, wherein the expandable frame is formed from a degradable material, and the degradable material degrades over time to release the monitor housing.

14. The monitor of claim 1, further comprising a telemetry unit to periodically transmit signals indicative of the sensed conditions over a period of time.

15. A monitor for placement within a urinary tract of a patient, the monitor comprising:
a monitor housing sized for introduction into a urethra of a patient;
a fixation structure to secure the device housing at a position within the urinary tract; and
a sensor to sense one or more physical characteristics of urine in the urinary tract.

16. The monitor of claim 15, wherein the physical characteristics include presence of drug residue in the urine.

17. The monitor of claim 15, wherein the physical characteristics include presence of at least one of sugar, proteins, blood, keytones, bilirubin, bacteria, yeast cells, and parasites in the urine.

18. The monitor of claim 15, wherein the physical characteristics include glucose level.

19. The monitor of claim 15, further comprising a telemetry unit to transmit signals indicative of the physical characteristics.

20. The monitor of claim 15, wherein the fixation structure includes an expandable frame.

21. The monitor of claim 20, wherein the expandable frame is degradable.

22. The monitor of claim 15, wherein the fixation structure includes a cavity formed in the monitor housing and a shaft to capture tissue within the cavity.

23. The monitor of claim 22, wherein the cavity includes a vacuum port for application of vacuum pressure to draw the tissue into the cavity.

24. The monitor of claim 22, wherein the shaft is sharpened to penetrate the tissue.

25. The monitor of claim 22, wherein the shaft is degradable.

26. The monitor of claim 15, further comprising a power source to power the sensor over a period of at least twenty-four hours.

27. The monitor of claim 15, further comprising a telemetry unit to periodically transmit signals indicative of the sensed conditions over a period of time.

28. A monitor for placement within a urinary tract of a patient, the monitor comprising:
a monitor housing sized for introduction into a urethra of a patient;
a fixation structure to secure the device housing at a position within the urethra; and
a sensor to sense one or more physiological conditions within the urinary tract.

29. The monitor of claim 28, wherein the sensor is configured to sense one or more urodynamic parameters.

30. The monitor of clam 29, wherein the urodynamic parameters includes at least one of urine pressure, urine flow, urine pH, temperature, and bladder contraction.

31. The monitor of claim 28, wherein the sensor is configured to sense one or more physical characteristics of urine in the urinary tract.

32. The monitor of claim 31, wherein the physical characteristics include presence of drug residue in the urine.

33. The monitor of claim 31, wherein the physical characteristics include presence of at least one of sugar, proteins, blood, keytones, bilirubin, bacteria, yeast cells, and parasites in the urine.

34. The monitor of claim 31, wherein the physical characteristics include glucose level.

35. The monitor of claim 28, further comprising a telemetry unit to transmit signals indicative of the sensed conditions.

36. The monitor of claim 28, wherein the fixation structure includes an expandable frame.

37. The monitor of claim 36, wherein the expandable frame is degradable.

38. The monitor of claim 28, wherein the fixation structure includes a cavity formed in the monitor housing and a shaft to capture tissue within the cavity.

39. The monitor of claim 38, wherein the cavity includes a vacuum port for application of vacuum pressure to draw the tissue into the cavity.

40. The monitor of claim 28, wherein the shaft is sharpened to penetrate the tissue.

41. The monitor of claim 28, wherein the shaft is degradable.

42. The monitor of claim 28, further comprising a power source to power the sensor over a period of at least twenty-four hours.

43. The monitor of claim 28, further comprising a telemetry unit to periodically transmit signals indicative of the sensed conditions over a period of time.

44. A system for monitoring a urinary tract of a patient, the monitor comprising:
a monitor including a monitor housing sized for introduction into a urethra of a patient, a fixation structure to secure the device housing at a position within the urinary tract, a sensor to sense one or more physiological conditions within the urinary tract, and a telemetry unit to transmit signals indicative of the sensed conditions; and
an external receiver to receive the transmitted signals and generate information based on the sensed conditions.

45. The system of claim 44, wherein the sensor is configured to sense one or more urodynamic parameters.

46. The system of clam 45, wherein the urodynamic parameters includes at least one of urine pressure, urine flow, urine pH, temperature, and bladder contraction.

47. The system of claim 44, wherein the sensor is configured to sense one or more physical characteristics of urine in the urinary tract.

48. The system of claim 47, wherein the physical characteristics include presence of drug residue in the urine.

49. The system of claim 47, wherein the physical characteristics include presence of at least one of sugar, proteins, blood, keytones, bilirubin, bacteria, yeast cells, and parasites in the urine.

50. The system of claim 47, wherein the physical characteristics include glucose level.

51. The system of claim 44, wherein the fixation structure includes an expandable frame mounted to the device housing to secure the device housing at a position within the urinary tract.

52. The system of claim 44, wherein the fixation structure includes a cavity formed in the monitor housing and a shaft to capture tissue within the cavity.

53. The system of claim 44, wherein the external receiver includes a display to present information based on the received signals.

54. The system of claim 44, wherein the information includes indications of detection of one or more of the characteristics.

55. The system of claim 44, wherein the information includes levels of one or more of the characteristics.

56. The system of claim 44, wherein the information includes one or more therapy recommendations.

57. The system of claim 44, wherein the telemetry unit transmits the signals periodically over a period of time, thereby updating the indication of the sensed conditions.

58. The system of claim 44, wherein the external receiver includes a display to present information based on the received signals, the system further comprising:
a network server communicatively coupled to the external receiver to receive the information; and
one or more network clients communicatively coupled to the network server to view the information.

59. The system of claim 44, wherein the external receiver includes a portable receiver housing for portability by the patient.

60. The system of claim 44, further comprising a therapy device to deliver therapy to the patient, wherein the therapy device is responsive to one of the external receiver and the monitor to adjust the delivered therapy in response to the sensed conditions.

61. The system of claim 60, wherein the therapy device includes one of a neurostimulator and a drug pump.

62. A method for monitoring a urinary tract of a patient, the method comprising:
introducing an endoscopic delivery device into a urethra of a patient;
positioning a monitor within the urinary tract of the patient with the endoscopic delivery device;
securing the monitor to tissue within the urinary tract with an expandable frame;
sensing one or more physiological conditions within the urinary tract via a sensor in the monitor; and
transmitting signals indicative of the sensed conditions from the sensor to an external receiver.

63. The method of claim 62, wherein the sensor is configured to sense one or more urodynamic parameters.

64. The method of clam 63, wherein the urodynamic parameters includes at least one of urine pressure, urine flow, urine pH, temperature, and bladder contraction.

65. The method of claim 62, wherein the sensor is configured to sense one or more physical characteristics of urine in the urinary tract.

66. The method of claim 65, wherein the physical characteristics include presence of drug residue in the urine.

67. The method of claim 65, wherein the physical characteristics include presence of at least one of sugar, proteins, blood, keytones, bilirubin, bacteria, yeast cells, and parasites in the urine.

68. The method of claim 65, wherein the physical characteristics include glucose level.

69. The method of claim 62, further comprising presenting information based on the received signals on a display.

70. The method of claim 69, wherein the information includes indications of detection of one or more of the characteristics.

71. The method of claim 69, wherein the information includes levels of one or more of the characteristics.

72. The method of claim 69, wherein the information includes one or more therapy recommendations.

73. The method of claim 62, further comprising transmitting the signals periodically over a period of time, thereby updating the indication of the sensed conditions.

74. A method for monitoring a urinary tract of a patient, the method comprising:
introducing an endoscopic delivery device into a urethra of a patient;
positioning a monitor within the urinary tract of the patient with the endoscopic delivery device;
securing the monitor to tissue within the urinary tract with an expandable frame;
sensing one or more physical characteristics of urine in the urinary tract via a sensor in the monitor; and
transmitting signals indicative of the sensed conditions from the sensor to an external receiver.

75. The method of claim 74, wherein the physical characteristics include presence of drug residue in the urine.

76. The method of claim 74, wherein the physical characteristics include presence of at least one of sugar, proteins, blood, keytones, bilirubin, bacteria, yeast cells, and parasites in the urine.

77. The method of claim 74, wherein the physical characteristics include glucose level.

78. The method of claim 74, further comprising presenting information based on the received signals on a display.

79. The method of claim 78, wherein the information includes indications of detection of one or more of the characteristics.

80. The method of claim 78, wherein the information includes levels of one or more of the characteristics.

81. The method of claim 78, wherein the information includes one or more therapy recommendations.

82. The method of claim 74, further comprising transmitting the signals periodically over a period of time, thereby updating the indication of the sensed conditions.

83. A monitor for placement within a urinary tract of a patient, the monitor comprising:
a monitor housing sized for introduction into a urethra of a patient;
means for securing the device housing at a position within the urinary tract; and
means for sensing one or more physical characteristics of urine in the urinary tract.

84. The monitor of claim 83, wherein the physical characteristics include presence of drug residue in the urine.

85. The monitor of claim 83, wherein the physical characteristics include presence of at least one of sugar, proteins, blood, keytones, bilirubin, bacteria, yeast cells, and parasites in the urine.

86. The monitor of claim 83, wherein the physical characteristics include glucose level.

87. The monitor of claim 83, further comprising means for transmitting signals indicative of the physical characteristics.
